(19) United States
(12) Patent Application Publication
(10) Pub. No.: US 2024/0033169 A1
(43) Pub. Date: Feb. 1, 2024

(54) MASSAGE DEVICE

(71) Applicant: Jeffrey William Domenighini, Phoenix, AZ (US)

(72) Inventor: Jeffrey William Domenighini, Phoenix, AZ (US)

(21) Appl. No.: 18/226,150

(22) Filed: Jul. 25, 2023

Related U.S. Application Data
(60) Provisional application No. 63/394,284, filed on Aug. 1, 2022.

(51) Int. Cl. A61H 19/00; A61B 10/00

(52) U.S. Cl. CPC A61H 19/32; A61B 10/0058

(57) ABSTRACT

An assembly comprising an elastic sleeve defining an inner cavity, the elastic sleeve comprising an outer surface and inner surface. The outer surface and inner surface extending from a distal end to a proximal end, the distal end in a closed position and the proximal end defining an opening. a lubricant provided within the inner cavity of the elastic sleeve. A sealing mechanism for providing a liquid seal that seals both the sleeve and the lubricant into a sealed assembly. A plurality of ribs may be provided along the inner surface of the elastic sleeve.

### PRIORITY CLAIM

This non-provisional patent application claims the benefit of U.S. Provisional Application No. 63/394,284 filed on Aug. 1, 2022, the entirety of which is incorporated herein by reference.

### FIELD

The present disclosure relates generally to massage devices. More particularly, the present disclosure relates to a massage device that functions as a sperm collection device.

### BACKGROUND

Message devices, like the presently disclosed sperm collection devices, may be used for medical research, for medical treatment, or simply for pleasure. That is, it is configured to collect sperm that is ejaculated from a man's penis. There is a need for a massage device that incorporates both an elastic sleeve as well as a lubricant in a sealed assembly.

### SUMMARY

According to an exemplary arrangement, an assembly comprises an elastic sleeve defining an inner cavity. The elastic sleeve comprises an outer surface and inner surface. The outer surface and the inner surface extending from a distal end to a proximal end of the elastic sleeve. The distal end resides in a closed position and the proximal end defining an opening. A lubricant is provided within the inner cavity of the elastic sleeve. A sealing mechanism provides a liquid seal that seals both the sleeve and the lubricant into a sealed assembly.

According to an exemplary arrangement, the assembly may comprise a plurality of ribs that are provided along the inner surface of the elastic sleeve. In an arrangement, the plurality of ribs provided along the inner surface of the elastic sleeve are equally spaced from one another. In one arrangement, the plurality of ribs comprise a plurality of circular ribs.

In one arrangement, at least one rib is provided along the outer surface of the elastic sleeve. For example, this at least one rib may be provided along the outer surface near the proximal end of the elastic sleeve.

According to an exemplary arrangement, the distal end of the elastic sleeve defines a sperm retaining chamber.

In one arrangement, the elastic sleeve comprises an elastomer.

In one arrangement, the lubricant comprises a water-based mixture.

In one arrangement, the assembly further comprises a resealable pouch configured to releasably contain the assembly. For example, in one arrangement, the resealable pouch comprises a plastic liner that facilitates sliding the assembly into a cavity defined by the releasable pouch. In one arrangement, the resealable pouch comprises a thermoplastic. In one arrangement, the releasable pouch comprises a plastic layer and a metal foil.

In one arrangement, the sealing mechanism of the assembly comprises a clip. For example, the clip may comprise a main body extending from a first end to a second end. The first end of the main body comprising a hook. An arm is pivotally connected to the first end of the main body, wherein the pivoting arm can be removably latched to the hook. In one arrangement, the clip comprises a metallic clip. In one arrangement, the clip comprises a thermoplastic clip.

In one arrangement, the sealing mechanism comprises an adhesive film. In such an arrangement, the adhesive film is sealed to provide the liquid seal that seals both the sleeve and the lubricant into the sealed assembly. For example, the adhesive film is heat-sealed to provide the liquid seal that seals both the sleeve and the lubricant into the sealed assembly. As another example, the adhesive film is press sealed to provide the liquid seal that seals both the sleeve and the lubricant into the sealed assembly. In one arrangement, the adhesive film comprises a thermoplastic film.

In one arrangement, the sealing mechanism comprises a shell case, the shell case comprising a first component hingedly attached to a second component. In one arrangement, the shell case comprises a thermoplastic. In one arrangement, the first component comprises a handle that allows the shell case to be moved from a closed position to an open position. In one arrangement, the assembly further comprises at least one snap hook, the at least one snap hook enabling the first component to be removably connected to the second component. In another arrangement, the assembly further comprises at least one press fit, the at least one press fit enabling the first component to be removably connected to the second component. In yet another arrangement, the assembly further comprises at least one ridge, the at least one ridge providing the liquid seal that seals both the sleeve and the lubricant into a sealed assembly.

In one arrangement, an assembly comprises an elastic sleeve defining an inner cavity, the elastic sleeve comprising an outer surface and inner surface, the outer surface and inner surface extending from a distal end to a proximal end, the proximal end in an open position; and a lubricant pouch provided within the inner cavity of the elastic sleeve. The lubricant pouch provides a liquid seal that seals a lubricant member into a sealed assembly within the inner cavity of the elastic sleeve. In one arrangement, the lubricant pouch comprises an extension portion that extends through the distal end of the elastic sleeve.

The features, functions, and advantages can be achieved independently in various embodiments of the present disclosure or may be combined in yet other embodiments in which further details can be seen with reference to the following description and drawings.

### BRIEF DESCRIPTION OF THE DRAWINGS

The novel features believed characteristic of the illustrative embodiments are set forth in the appended claims. The illustrative embodiments, however, as well as a preferred mode of use, further objectives and descriptions thereof, will best be understood by reference to the following detailed description of one or more illustrative embodiments of the present disclosure when read in conjunction with the accompanying drawings, wherein:

FIG. 1 illustrates multiple perspective views of an elastic sleeve for use with an assembly, according to an example embodiment;

FIG. 2A illustrates a view of a sealing mechanism for use with elastic sleeve, such as the elastic sleeve illustrated in FIG. 1,

FIG. 2B illustrates an alternative view of a sealing mechanism for use with elastic sleeve, such as the elastic sleeve illustrated in FIG. 1,

FIG. 3 illustrates a first elastic sleeve and an open sealing mechanism;

FIG. 4 illustrates a first elastic sleeve and a partially closed sealing mechanism;

FIG. 5 illustrates a first elastic sleeve and a closed sealing mechanism;

FIG. 6 illustrates perspective view of a resealable package for use with an elastic sleeve, such as the elastic sleeve illustrated in FIG. 1;

FIG. 7 illustrates a perspective view of a sealing mechanism for use with an elastic sleeve, such as the elastic sleeve illustrated in FIG. 1;

FIG. 8 illustrates a perspective a sealing mechanism for use with elastic sleeve, such as the elastic sleeve illustrated in FIG. 1;

FIG. 9 illustrates a perspective view of an elastic sleeve for use with an assembly, according to an example embodiment

FIGS. 10 and 11 illustrate perspective views of an elastic sleeve for use with an assembly, according to another example embodiment; and

FIG. 12 illustrates a perspective a sealing mechanism for use with elastic sleeve, such as the elastic sleeve illustrated in FIG. 1.

### DETAILED DESCRIPTION

The following detailed description describes various features and functions of the disclosed systems and methods with reference to the accompanying figures. The illustrative system and method embodiments described herein are not meant to be limiting. It may be readily understood that certain aspects of the disclosed systems and methods can be arranged and combined in a wide variety of different configurations, all of which are contemplated herein.

Further, unless the context suggests otherwise, the features illustrated in each of the figures may be used in combination with one another. Thus, the figures should be generally viewed as component aspects of one or more overall implementations, with the understanding that not all illustrated features are necessary for each implementation.

Additionally, any enumeration of elements, blocks, or steps in this specification or the claims is for purposes of clarity. Thus, such enumeration should not be interpreted to require or imply that these elements, blocks, or steps adhere to a particular arrangement or are carried out in a particular order.

By the term “substantially” it is meant that the recited characteristic, parameter, or value need not be achieved exactly, but that deviations or variations, including for example, tolerances, measurement error, measurement accuracy limitations and other factors that are known to those of ordinary skill in the art, may occur in amounts that do not preclude the effect the characteristic was intended to provide.

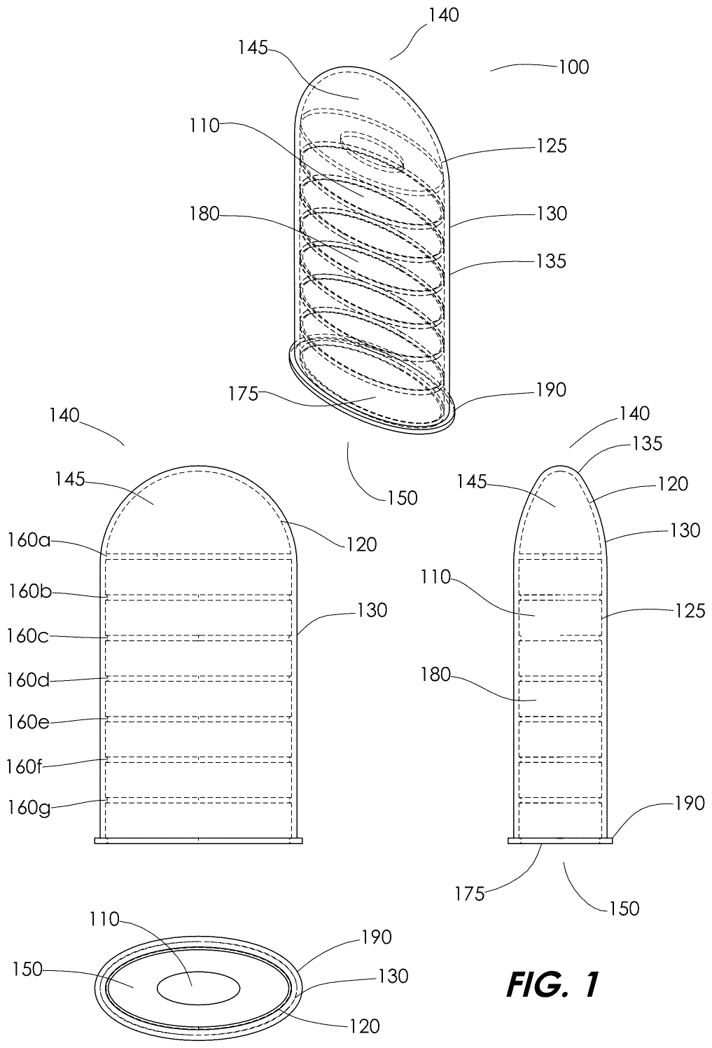

FIG. 1 illustrates various perspective views of a sperm collection device or elastic sleeve 100 according to one arrangement. As illustrated, this sperm collection device 100 takes the form of an assembly comprising an elastic sleeve 100 wherein this elastic sleeve 100 defines an inner cavity 110. Different types of elastic materials may be used to form the elastic sleeve 100. For example, the elastic sleeve may comprise an elastomer like Thermoplastic elastomers (TPE), sometimes referred to as thermoplastic rubbers (TPE), thermoplastic polyurethane (TPU), or silicone.

In addition, the elastic sleeve 100 comprises an outer surface 130 along with an inner surface 120. In one preferred arrangement, the inner surface 120 may comprise a smooth inner surface 125. In one arrangement, the outer surface 130 and the inner surface 120 may comprise a slightly rough or texturized surface (e.g., sandblasted) so as to enhance gripping along the outer surface 130 and so as to enhance sensation along the inner surface 120.

In one preferred arrangement, the outer surface 130 comprises a smooth outer surface 135. However, those of ordinary skill in the art will recognize, that alternative inner surfaces, outer surfaces, structures, and configurations may also be used.

As illustrated, the outer surface 130 and inner surface 120 extend from a distal end 140 of the elastic sleeve 100 to a proximal end 150 of the sleeve 100. In one arrangement, the distal end 140 comprises a closed position or sealed end position. In one preferred arrangement, the closed distal end 140 of the elastic sleeve 100 defines a sperm retaining chamber 145. This sperm retaining chamber 145 comprises a chamber or cavity that is configured to capture sperm that is ejaculated from a man's penis.

At the opposite end of the sleeve 100 or at the proximal end 150 of the elastic sleeve 100, the sleeve defines an opening 175. This opening 175 is designed so that a certain amount of lubricant 180 can be injected into or placed within the inner cavity 110 defined by the elastic sleeve 100. In addition, this opening 175 is designed to elastically receive an erect penis so that when the penis ejaculates, the sperm retaining chamber 145 of the elastic sleeve 100 will contain any sperm that is ejaculated.

In addition, the assembly further comprises a lubricant 180 that is provided within the inner cavity 110 of the elastic sleeve 100. As just one example, such a liquid lubricant 180 may operate as a lotion or pleasure enhancer. This liquid lubricant 180 may comprise a water-based mixture, an oil-based mixture, or a glycerin-based mixture. Other types of lubricants, lotions, or pleasure enhancers may also be utilized. In one arrangement, approximately 3 to about 5 ml of lubricant 180 is placed within the inner cavity 110 of the elastic sleeve 100.

In one arrangement, a plurality of ribs 160a-g are provided along the inner surface 120 of the elastic sleeve 100. In the elastic sleeve arrangement 100 illustrated in FIG. 1, seven ribs 160 a, b, c, d, e, f, g are provided along this inner surface 120 of the elastic sleeve 100. However, in other types of elastic sleeve arrangements, more or less than seven ribs may be provided. In addition, as illustrated, the assembly comprises the plurality of ribs 160a-g provided along the inner surface 120 of the elastic sleeve 100 are equally spaced from one another. That is, the first rib 160a is spaced from the second rib 160b a similar distance as the third rib 160c is spaced from a fourth rib 160d. As illustrated, the plurality of ribs 160a-g comprise a plurality of circular ribs. In addition, in this illustrated rib configuration, all of the illustrated ribs comprise a similar dimension, that is similar height and/or width. However, ribs of different dimensions, structure and geometric configurations may also be utilized.

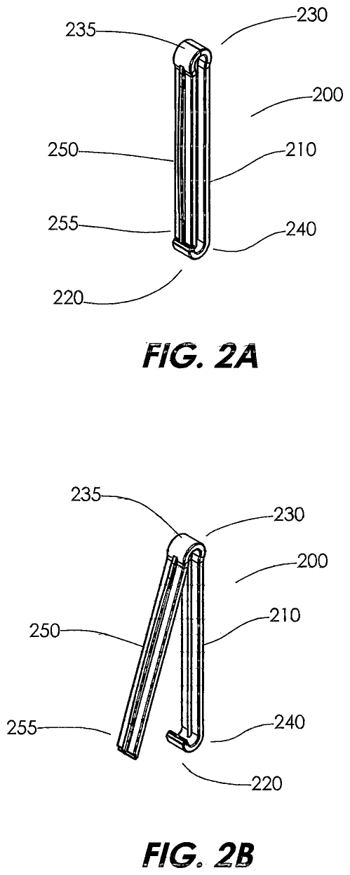

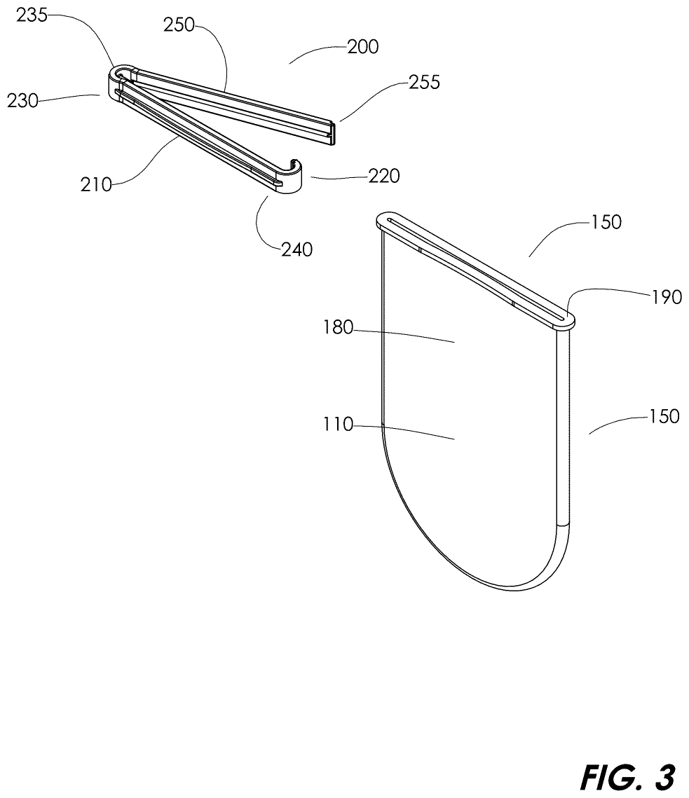

In addition, as illustrated, an external rib 190 is provided along the outer surface 130 of the elastic sleeve 100. As will be described, this outer external rib feature 190 is configured to engage a sealing mechanism (FIGS. 2-6) that will be utilized to removably secure to the proximal end 150 of the elastic sleeve 100. In one arrangement, more than one outer external rib feature 190 may be utilized.

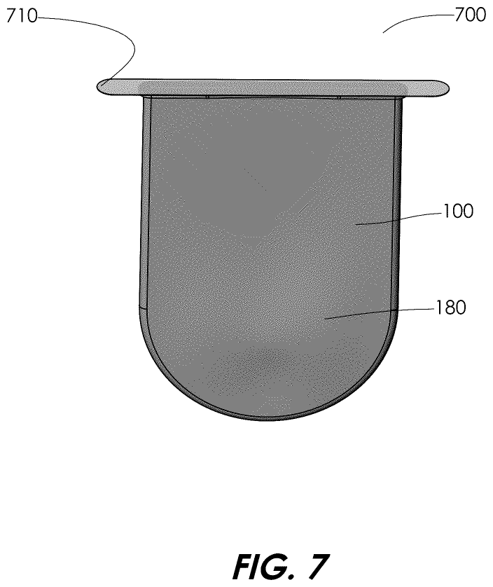

A resealable pouch may be used to transport the elastic sleeve 100 and its contained lubricant 180. In one preferred arrangement, the assembly may further comprise a resealable pouch 600 (FIG. 6) that is configured to releasably contain the assembly. For example, in one arrangement, the resealable pouch 600 may comprise a plastic liner 610 that facilitates sliding the assembly into a cavity 620 defined by the resealable pouch 600.

In one arrangement, the resealable pouch 600 comprises a thermoplastic or thermoplastic like material 610. In yet another arrangement, the resealable pouch 600 comprises a plastic layer 625 and a metal foil 630. A front surface 660 and/or a back surface 670 of the resealable pouch 600 may be provided with text and/or other inscriptions 650 regarding the contents of the resealable pouch 600, storage requirements, intended use (elastic sleeve instructions) as well as provider or supplier information.

FIGS. 2A and 2B provide various perspective views of a one arrangement of a sealing device 200 that may be used with the elastic sleeve 100 illustrated in FIG. 1 so as to form a massage device assembly. As illustrated, the assembly comprises a sealing device or sealing mechanism 200 for providing a liquid seal to the opening of an elastic sleeve, such as the elastic sleeve 100 illustrated in FIG. 1. Such a seal may seal both the elastic sleeve 100 and the lubricant 180 provided in a sealed assembly. Among other issues, this seal prevents the lubricant 180 from exiting the inner cavity 110 of the elastic sleeve 100 prior to use.

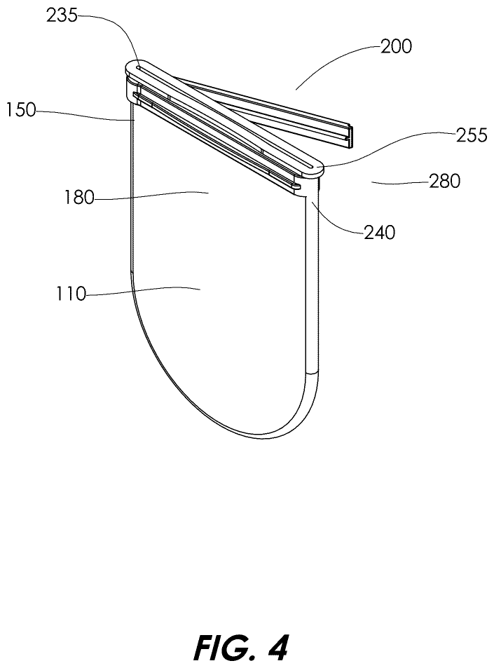

FIGS. 2A and 2B illustrate multiple views of one arrangement of a sealing device or mechanism 200 for use with the elastic sleeve 100 illustrated in FIG. 1. In this arrangement, the sealing mechanism 200 comprises a pivoting clip member. For example, FIG. 2A illustrates this sealing device 200 in a closed, a clasping, or sealing position while FIG. 2B illustrates this sealing device 200 in an open or unsealing position. FIG. 3 illustrates this sealing device 200 in an open position before the sealing device 200 is used to seal off the open proximal end 150 of the elastic sleeve 100.

Referring now to FIGS. 2A, B-5, the sealing mechanism 200 comprises a main body 210 that extends from a first end 220 of the clip 200 to a second end 230 of the clip 200. At the second end 230 of the main body 210, a pivot area 235 is provided. In this illustrated arrangement, the first end 220 of the main body 210 comprises a hook 240. In this sealing mechanism arrangement, a pivoting arm 250 is pivotally connected to the pivoting area 235 near the first end 230 of the main body 210 of the sealing mechanism 200. As illustrated in FIG. 3, the first end 220 of the main body 210 is uncoupled to the pivoting arm 250. Specifically, and as illustrated in FIG. 3, the first end 220 of the main body 210 is uncoupled to a first end 255 of the pivoting arm 250.

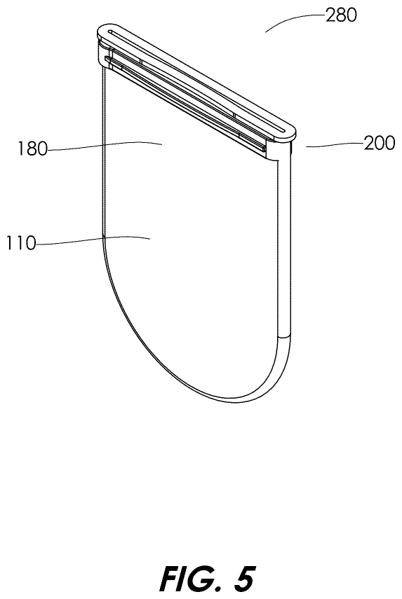

In use, the pivoting arm 250 of this illustrated sealing mechanism or device 200 can be removably latched to the hook element 240. In this manner and as illustrated in FIG. 4, when the clip 200 is provided near the external rib feature 190 located adjacent the proximal end 150 of the elastic sleeve 100, the sealing mechanism or device 200 will be releasably locked so as to provide a liquid seal to the elastic sleeve 100 and its contents contained within its inner cavity 110 (Le., the liquid lubricant 180). Such a seal may seal both the elastic sleeve 100 and the lubricant 180 into a sealed assembly.

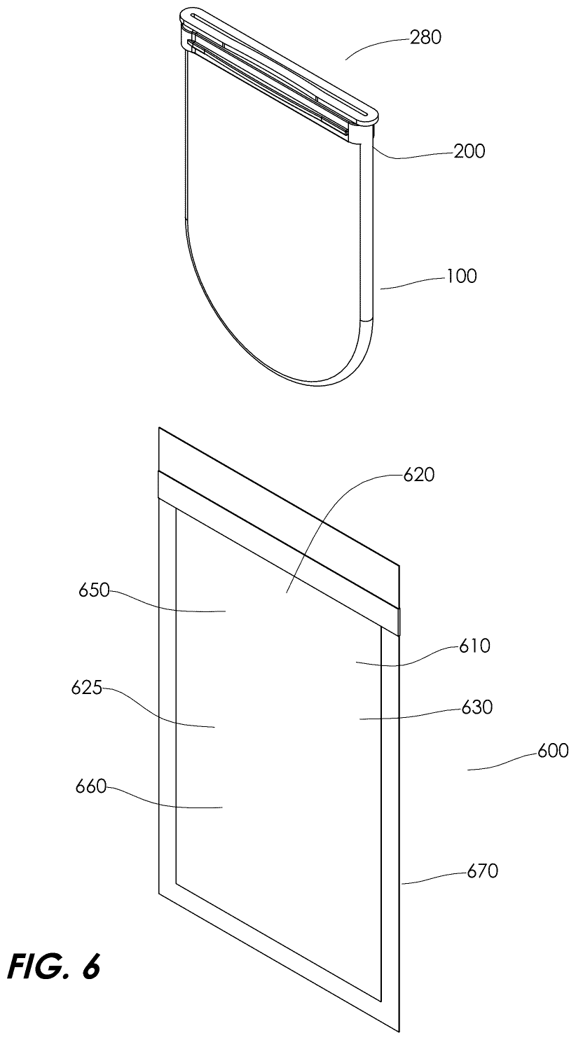

FIG. 5 illustrates the sealing device 200 in a closed position thereby sealing off the proximal end 150 of the elastic sleeve 100. This sealing action acts so as to form a sealed assembly arrangement 280. One advantage of using such a sealing mechanism 200 is that it prevents the lubricant 180 from exiting or escaping the inner cavity 110 of the elastic sleeve 100. Another advantage of such a sealing mechanism 200 is that it provides a reusable sealing mechanism, one that can be used multiple times.

Different types of clip configurations may be used, and these clips may comprise different types of materials. As just one example, in one arrangement, the clip 200 may comprise a metallic clip. In an alternative arrangement, the clip 200 may comprise a thermoplastic clip. As those of ordinary skill in the art will recognize, alternative clip materials and/or clip configurations may also be utilized. As just one example, an alternative clip may comprise a multi-component type of sealing mechanism. As yet another example, the sealing mechanism may comprise a one-time use mechanism which can no longer be used once it is removed from the assembly.

The assembly illustrated in FIG. 5 is now ready for transport or ready for commercial packaging. For example, FIG. 6 illustrates a final assembly read for insertion or packaging into a resealable pouch 600.

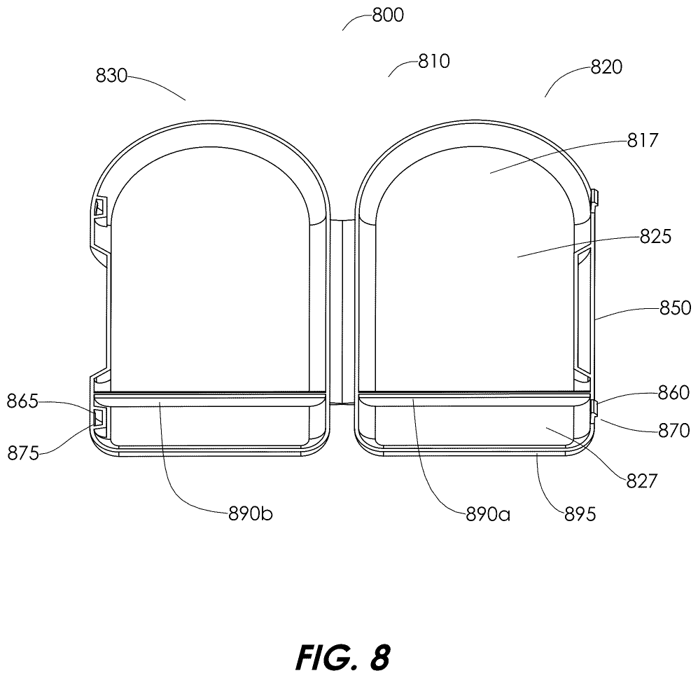

FIG. 7 illustrates an alternative arrangement of a sealing mechanism 700 for use with an elastic sleeve. Such a mechanism 700 may be used with an elastic sleeve, such as the elastic sleeve 100 illustrated in FIG. 1. As illustrated, the sealing mechanism 700 comprises an adhesive film 710. This adhesive film 710 may be sealed to provide the liquid seal that seals both the sleeve 100 and the lubricant 180 into the sealed assembly. As just one example, a heat seal or a pressure seal may be provided that allows for the adhesive film to seal both the sleeve and the lubricant into the sealed assembly. In one arrangement, the adhesive film 710 comprises a thermoplastic film. As those of ordinary skill in the art will recognize, alternative films may also be utilized.

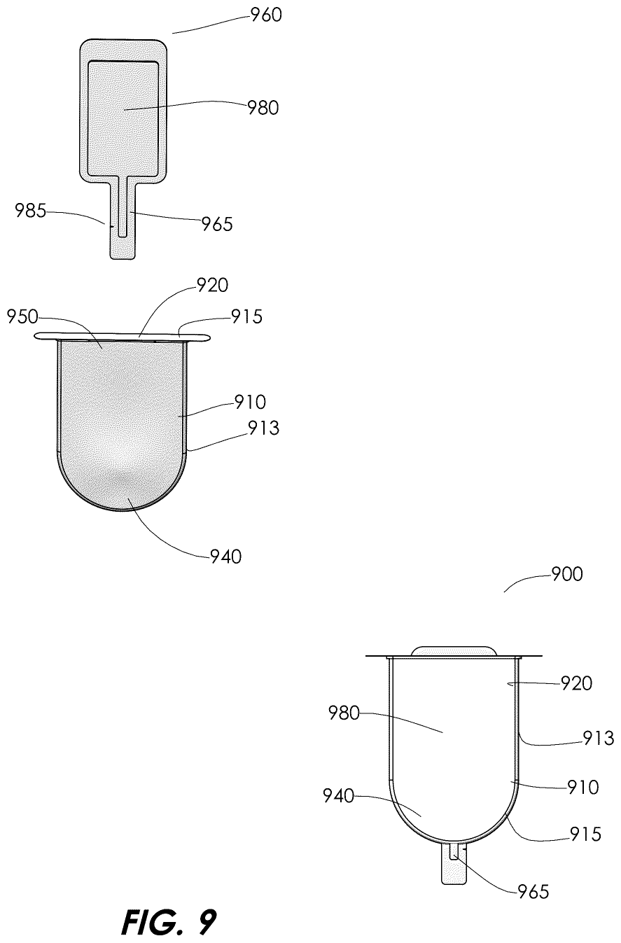

FIG. 8 illustrates an alternative arrangement of a sealing mechanism 800. Such a sealing mechanism 800 may be used with an elastic sleeve, such as the elastic sleeve 100 illustrated in FIG. 1. As illustrated in FIG. 8, the sealing mechanism 800 comprises a shell case 810. In this exemplary arrangement, the shell case 810 comprises a first component 820 that is pivotally or hingedly attached to a second component 830. This allows this sealing mechanism 800 to be repeatedly opened and closed so that access to an inner cavity defined by the sealing mechanism may be accessed. Both the first component 820 and the second component 830 are similarly shaped. In one arrangement, the shell case 810 comprises a thermoplastic. One advantage of such an arrangement is that the shell case 810 acts as an individual packaging for the elastic sleeve thereby providing a sealed enclosure for an elastic sleeve containing a lubricant.

Referring to FIG. 8, the first component 820 of the sealing mechanism 800 comprises a receiving cavity 825 for receiving an elastic sleeve, such as the elastic sleeve 100 illustrated in FIG. 1. As illustrated, this receiving cavity 825 comprises a first receiving end 817 for receiving a distal end of an elastic sleeve, like the distal end 140 of the elastic sleeve 100 illustrated in FIG. 1. In addition, the receiving cavity 825 comprises a second receiving end 827 for receiving an open proximal end of the elastic sleeve, like the proximal end 150 of the elastic sleeve 100 illustrated in FIG. 1.

In addition, the first component 820 also comprises at least one handle 850 that is configured so as to allow the shell case 810 to be moved from a closed position to an open position (as illustrated in FIG. 8). In addition, the shell case 810 is further configured to comprise at least one snap hook 860. This at least one snap hook 860 enables the first component 820 to be releasably connected to the second component 830. In one preferred arrangement, the at least one snap hook 860 of the first component 820 is configured to releasably engage at least one snap hook receptacle 865 provided by the second component 830.

In one arrangement, the sealing mechanism further comprises at least one press fit 870. This at least one press fit 870 enabling the first component 820 to be removably connected to the second component 830. In one preferred arrangement, the at least one press fit 870 of the first component 820 is configured to releasably engage at least one press fit receptacle 875 provided by the second component 830.

In one arrangement, the sealing mechanism further comprises at least one ridge 890. As illustrated, this at least one ridge 890 may be provided along a lower portion 895 of one or both of the first and second components 820, 830. For example, this at least one ridge may be provided near a bottom potion of the receiving cavity defined by the first component 820.

In this arrangement, when an elastic sleeve containing a lubricant (as illustrated in FIG. 1) is inserted into the receiving cavity 825 and then these two components 820, 830 are collapsed towards one another so that the shell 810 resides in a closed state, the at least one ridge 890a of the first component 820 and the at least one ridge 890b of the second component 830 will engage or press against the proximal end of the elastic sleeve to thereby form a liquid seal that seals both the sleeve 100 and the lubricant 180 into a sealed assembly.

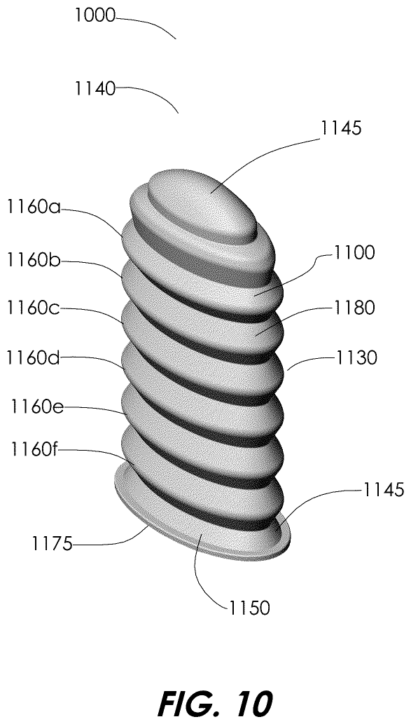

FIG. 9 illustrates perspective views of an alternative assembly 900. In this illustrated arrangement, the assembly 900 comprises an elastic sleeve 910 defining an inner cavity 920. This elastic sleeve may be similar to the elastic sleeve 100 illustrated in FIG. 1.

As illustrated in FIG. 9, this elastic sleeve 910 comprises an outer surface 913 and inner surface 915. The outer surface 913 and inner surface 915 extend from a distal end 940 to a proximal end 950, the proximal end 950 in an open position. In one preferred arrangement, the elastic sleeve 910 illustrated in FIG. 8 has many of the same material and design characteristics as the elastic sleeve 100 illustrated in FIG. 1.

The assembly 900 illustrated in FIG. 9 further comprises a separate lubrication container in the form of a lubricant pouch 960. In the assembled state, as illustrated in FIG. 9, this lubrication container 960 is provided within or inserted into the inner cavity 920 of the elastic sleeve 910. In one preferred arrangement, this lubricant pouch 960 is configured to releasably contain between about 3 to about 5 ml of a lubricant 980. The lubricant pouch 960 provides a liquid seal that seals the lubricant within the sealed assembly within the inner cavity 920 of the elastic sleeve 910.

As illustrated, the lubricant pouch 960 comprises an extension portion or a tail 965 that extends from the distal end 985 of the lubricant pouch 960. After the lubricant pouch 960 has been placed or inserted within the inner cavity 920 defined by the elastic sleeve 910, the extension portion or tail 965 of the lubricant pouch 960 will extend through an opening defined within the distal 940 end of the elastic sleeve 910.

In use, a user will tear the extension portion 965 of the lubricant pouch 960 off. The user will then pull this lubrication container 960 out of the elastic sleeve 910 with one hand, while the user will use the other hand to pinch or squeeze the elastic sleeve 910 near the middle of the body of the lubricant pouch. By pinching along this area of the elastic sleeve 910, the user will then squeeze the liquid lubricant 980 out of the pouch 960 and the onto the inner walls of the elastic sleeve 910.

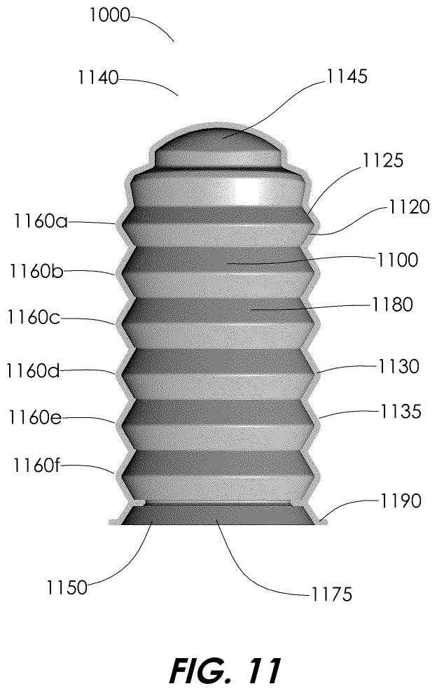

FIGS. 10 and 11 illustrate perspective views of a sperm collection device or elastic sleeve 1000 according to one arrangement. As illustrated, this sperm collection device 1000 takes the form of an assembly comprising an elastic sleeve 1000 wherein this elastic sleeve 1000 defines an inner cavity 1100. Different types of elastic materials may be used to form the elastic sleeve 1000. For example, the elastic sleeve may comprise an elastomer like Thermoplastic elastomers (TPE), sometimes referred to as thermoplastic rubbers (TPE), thermoplastic polyurethane (TPU), or silicone.

In addition, the elastic sleeve 1000 comprises an outer surface 1130 along with an inner surface 1120. In one preferred arrangement, the inner surface 1120 may comprise a smooth inner surface 1250. In one arrangement, the outer surface 1300 and the inner surface 120 may comprise a slightly rough or texturized surface (e.g., sandblasted) so as to enhance gripping along the outer surface 1130 and so as to enhance sensation along the inner surface 1120.

In one preferred arrangement, the outer surface 1130 comprises a smooth outer surface 1135. However, those of ordinary skill in the art will recognize, that alternative inner surfaces, outer surfaces, structures, and configurations may also be used.

As illustrated, the outer surface 1130 and inner surface 1120 extend from a distal end 1140 of the elastic sleeve 1000 to a proximal end 1150 of the sleeve 1000. In one arrangement, the distal end 1140 comprises a closed position or sealed end position. In one preferred arrangement, the closed distal end 1140 of the elastic sleeve 1000 defines a sperm retaining chamber 1145. This sperm retaining chamber 1145 comprises a chamber or cavity that is configured to capture sperm that is ejaculated from a man's penis.

At the opposite end of the sleeve 1000 or at the proximal end 1150 of the elastic sleeve 1000, the sleeve defines an opening 1175. This opening 1175 is designed so that a certain amount of lubricant 1180 can be injected into or placed within the inner cavity 1100 defined by the elastic sleeve 1000. In addition, this opening 1175 is designed to elastically receive an erect penis so that when the penis ejaculates, the sperm retaining chamber 1145 of the elastic sleeve 1000 will contain any sperm that is ejaculated.

In addition, the assembly further comprises a lubricant 1180 that is provided within the inner cavity 1100 of the elastic sleeve 1000. As just one example, such a liquid lubricant 1180 may operate as a lotion or pleasure enhancer. This liquid lubricant 1180 may comprise a water-based mixture, an oil-based mixture, or a glycerin-based mixture. Other types of lubricants, lotions, or pleasure enhancers may also be utilized. In one arrangement, approximately 3 to about 5 ml of lubricant 1180 is placed within the inner cavity 1100 of the elastic sleeve 1000.

In one arrangement, a plurality of sinusoidal wavelets or undulations 1600a-g are provided along the inner surface 1200 of the elastic sleeve 1000. In the elastic sleeve arrangement 1000 illustrated in FIGS. 10 and 11, six wavelets 1160 a, b, c, d, e, f are provided along the elastic sleeve 1000. However, in other types of elastic sleeve arrangements, more or less than six wavelets may be provided. In addition, as illustrated, the assembly comprises the plurality of wavelets 1160 a-f provided along the elastic sleeve 1000 are equally spaced from one another. That is, the first wavelet 1160a is spaced from the second wavelet 1160b a similar distance as the third wavelet 1160c is spaced from a fourth wavelet 1160d. As illustrated, the plurality of wavelets 1160 a-f comprise a plurality of circular wavelets. In addition, in this illustrated configuration, all of the illustrated wavelets comprise a substantially similar dimension, that is similar height and/or width. However, wavelets of different dimensions, structure and geometric configurations may also be utilized.

In addition, as illustrated, an external rib 1190 is provided along the outer surface 1130 of the elastic sleeve 1000. As will be described, this outer external rib feature 1190 is configured to engage a sealing mechanism (FIGS. 2-6) that will be utilized to removably secure to the proximal end 1150 of the elastic sleeve 1000. In one arrangement, more than one outer external rib feature 1190 may be utilized.

A resealable pouch may be used to transport the elastic sleeve 1000 and its contained lubricant 1180. In one preferred arrangement, the assembly may further comprise a resealable pouch 600 (FIG. 6) that is configured to releasably contain the assembly. For example, in one arrangement, the resealable pouch 600 may comprise a plastic liner 610 that facilitates sliding the assembly into a cavity 620 defined by the resealable pouch 600.

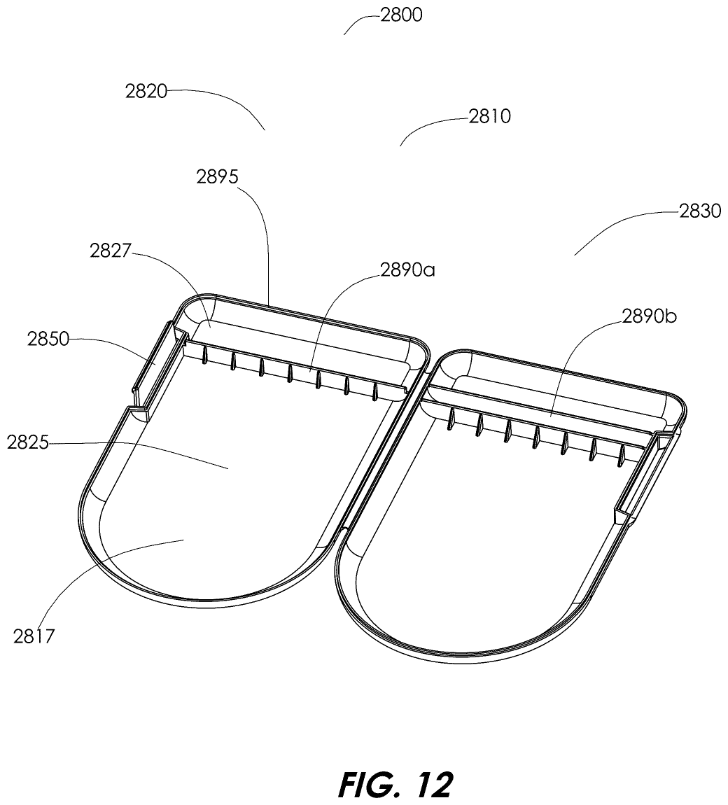

FIG. 12 illustrates an alternative arrangement of a sealing mechanism 2800. Such a sealing mechanism 2800 may be used with an elastic sleeve, such as the elastic sleeves illustrated in FIGS. 1 and 10, 11. As illustrated in FIG. 12, the sealing mechanism 2800 comprises a shell case 2810. In this exemplary arrangement, the shell case 2810 comprises a first component 2820 that is pivotally or hingedly attached to a second component 2830. This allows this sealing mechanism 2800 to be repeatedly opened and closed so that access to an inner cavity defined by the sealing mechanism may be accessed. Both the first component 2820 and the second component 2830 are similarly shaped. In one arrangement, the shell case 2810 comprises a thermoplastic. One advantage of such an arrangement is that the shell case 2810 acts as an individual packaging for the elastic sleeve thereby providing a sealed enclosure for an elastic sleeve containing a lubricant.

Referring to FIG. 12, the first component 2820 of the sealing mechanism 2800 comprises a receiving cavity 2825 for receiving an elastic sleeve, such as the elastic sleeves illustrated in FIGS. 1 and 10, 11. As illustrated, this receiving cavity 2825 comprises a first receiving end 2817 for receiving a distal end of an elastic sleeve, like the distal end 140 of the elastic sleeve 100 illustrated in FIG. 1 or the distal end 1140 of the elastic sleeve 1000 illustrated in FIGS. 10 and 11. In addition, the receiving cavity 2825 comprises a second receiving end 2827 for receiving an open proximal end of the elastic sleeve, like the proximal end 150 of the elastic sleeve 100 illustrated in FIG. 1 or the proximal end 1150 of the elastic sleeve 1000 illustrated in FIGS. 10 and 11.

In addition, the first component 2820 also comprises at least one handle 2850 that is configured so as to allow the shell case 2810 to be moved from a closed position to an open position (as illustrated in FIG. 12).

In one arrangement, the sealing mechanism further comprises at least one ridge 2890 a,b. As illustrated, this at least one ridge 2890 may be provided along a lower portion 2895 of one or both of the first and second components 2820, 2830. For example, this at least one ridge may be provided near a bottom potion of the receiving cavity defined by the first component 2820.

In this arrangement, when an elastic sleeve containing a lubricant (as illustrated in FIG. 1) is inserted into the receiving cavity 2825 and then these two components 2820, 2830 are collapsed towards one another so that the shell 2810 resides in a closed state, the at least one ridge 2890a of the first component 2820 and the at least one ridge 2890b of the second component 2830 will engage or press against the proximal end of the elastic sleeve to thereby form a liquid seal that seals both the sleeve 100 (or sleeve 1000) and the lubricant 180 into a sealed assembly.

The description of the different advantageous embodiments has been presented for purposes of illustration and description and is not intended to be exhaustive or limited to the embodiments in the form disclosed. Modifications and variations will be apparent to those of ordinary skill in the art. Further, different advantageous embodiments may provide different advantages as compared to other advantageous embodiments. The embodiment or embodiments selected are chosen and described in order to best explain the principles of the embodiments, the practical application, and to enable others of ordinary skill in the art to understand the disclosure for various embodiments with various modifications as are suited to the particular use contemplated.

I claim:

1. An assembly comprising:
an elastic sleeve defining an inner cavity, the elastic sleeve comprising an outer surface and inner surface, the outer surface and inner surface extending from a distal end to a proximal end, the distal end in a closed position and the proximal end defining an opening;
a lubricant provided within the inner cavity of the elastic sleeve; and
a sealing mechanism for providing a liquid seal that seals both the sleeve and the lubricant into a sealed assembly.

2. The assembly of claim 1 wherein a plurality of ribs are provided along the inner surface of the elastic sleeve.

3. The assembly of claim 1 wherein a rib is provided along the outer surface of the elastic sleeve.

4. The assembly of claim 1 wherein the distal end of the elastic sleeve defines a sperm retaining chamber.

5. The assembly of claim 1 wherein the elastic sleeve comprises an elastomer.

6. The assembly of claim 1 wherein the lubricant comprises a water based mixture.

7. The assembly of claim 1 further comprising a resealable pouch configured to releasably contain the assembly.

8. The assembly of claim 1 wherein the sealing mechanism comprises a clip, the clip comprising
a main body extending from a first end to a second end, the first end of the main body comprising a hook, and
an arm pivotally connected to the first end of the main body, wherein the pivoting arm can be removably latched to the hook.

9. The assembly of claim 1 wherein the clip comprises a metallic clip.

10. The assembly of claim 1 wherein the clip comprises a thermoplastic clip.

11. The assembly of claim 1 wherein the sealing mechanism comprises an adhesive film, wherein the adhesive film is heat sealed to provide the liquid seal that seals both the sleeve and the lubricant into the sealed assembly.

12. The assembly of claim 11 wherein the adhesive film comprises a thermoplastic film.

13. The assembly of claim 1 wherein the sealing mechanism comprises a shell case, the shell case comprising a first component hingedly attached to a second component.

14. The assembly of claim 13 wherein the shell case comprises a thermoplastic.

15. The assembly of claim 14 wherein the first component comprises a handle that allows the shell case to be moved from a closed position to an open position.

16. The assembly of claim 15 further comprising at least one snap hook, the at least one snap hook enabling the first component to be removably connected to the second component.

17. The assembly of claim 16 further comprising at least one press fit, the at least one press fit enabling the first component to be removably connected to the second component.

18. The assembly of claim 17 further comprising at least one ridge, the at least one ridge providing the liquid seal that seals both the sleeve and the lubricant into a sealed assembly.

19. The assembly of claim 1 wherein a plurality of wavelets are provided along the inner surface of the elastic sleeve.

20. An assembly comprising:
an elastic sleeve defining an inner cavity, the elastic sleeve comprising an outer surface and inner surface, the outer surface and inner surface extending from a distal end to a proximal end, the proximal end in an open position; and
a lubricant pouch provided within the inner cavity of the elastic sleeve;
wherein the lubricant pouch provides a liquid seal that seals a lubricant member into a sealed assembly within the inner cavity of the elastic sleeve.
